(19) United States
(12) Patent Application Publication
(10) Pub. No.: US 2006/0223827 A1
(43) Pub. Date: Oct. 5, 2006

(54) Bioreductively activated stilbene prodrugs

(75) Inventors: Peter David Davis, Oxford (GB); Matthew Alexander Naylor, Northwood Middlesex (GB); Peter Thomson, Northwood Middlesex (GB); Steven Albert Everett, Northwood Middlesex (GB); Michael Richard Lacey Stratford, Northwood Middlesex (GB); Peter Wardman, Northwood Middlesex (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Siemens Aktiengesellschaft

(21) Appl. No.: 10/550,982

(22) PCT Filed: Mar. 26, 2004
(86) PCT No.: PCT/GB04/01341

§ 371 Date: Oct. 27, 2005

(30) Foreign Application Priority Data
Mar. 26, 2003 (GB) 0306908.5

(51) Int. Cl. A61K 31/496; A61K 31/404; A61K 31/381; C07D 333/22; C07D 403/02; C07D 209/04

(52) U.S. Cl. 514/254.09; 514/447; 514/415; 514/682; 544/373; 549/76; 548/509; 552/292

(57) ABSTRACT

A compound of formula (1), or a pharmaceutically acceptable salt thereof, wherein Ar is a substituted heteroaryl group bearing at least one nitro or azido group or is a group of formula (2) or (3); R1 is hydrogen, optionally substituted alkyl, optionally substituted aryl or optionally substituted heteroaryl; R₂ is alkyl, alkoxy, thialkoxy or halo; R₃, R₄ and R₅ are each independently alkyl, alkoxy, thialkoxy or halo with the proviso that at least two of R₃, R₄ or R₅ are alkoxy; L is —OC(O)— or —OP(O)(OR₆)—; n is 0 or 1; X is O, S or NR₇; Y is hydrogen, alkyl, alkoxy, thialkoxy, halo, hydroxy or dihydrogenphosphate; R₆ is hydrogen or alkyl; R₇ is hydrogen or alkyl; R₈ is hydrogen, alkoxy or dialkylaminoalkyl; R₉ is optionally substituted alkyl; R₁₀ is hydrogen, alkyl, alkoxy or dialkylaminoalkyl; R₁₁, and R₁₂ are independently hydrogen, alkyl, alkoxy, thialkoxy, amono alkylamino, dialkylamino, morpholino, alkylmorpholino, piperidino, alkylpiperidiono, piperazino, alkylpiperazino or 1-aziridinyl; and A, together with the carbon atoms to which it is fused, is an optionally substituted aryl or heteroaryl ring.

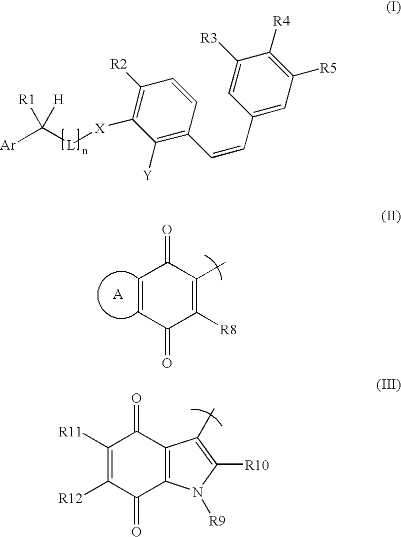

This invention relates to compounds useful in the treatment of cell proliferation disorders. More particularly the invention relates to a series of compounds that are activated under hypoxic conditions.

Many drugs used in conventional cancer chemotherapy are toxic to growing cancer cells but lack complete specificity. Thus other normal tissues are affected and ensuing side effects limit the dose that can be administered. Therefore the exposure of the cancerous tumour to the compound, and in turn the effectiveness of the therapy, is limited. Recent research has shown promising clinical activity of compounds, such as protein kinase inhibitors, which are cytostatic in their action. However the specificity of such compounds is not complete and side effects arising from action against normal tissues can again limit the effectiveness of therapy. There is a need for drugs that target the tumour more selectively.

Many solid tumours exhibit regions of hypoxia (low oxygen tension). Inadequate blood supply to the central regions of the tumour results in hypoxia that can be chronic or acute. This hypoxia represents a challenge to effective therapy by radiation or by conventional chemotherapy since hypoxic regions are often more resistant to these modalities. It has been suggested, however, that tumour hypoxia can be used to target tumours for drug action (Kennedy, Cancer Res. 1980, 40, 2356-2360.). One particular method of using the hypoxic regions of tumours for drug targeting is the selective activation of produgs under conditions of low oxygen tension. A concept has been advanced whereby the activity of a cytotoxic compound can be masked by a trigger moiety which, under hypoxic conditions, mediates fragmentation of the masked cytotoxic compound into the active cytotoxic agent (Denny, Lancet Oncol 2000, 1, 25-9). Compounds attempting to utilize this concept typically consist of the trigger moiety attached, often via a linker moiety, to a cytotoxic moiety (the effector).

Hypoxia is also a feature of the rheumatoid arthritic joint (Rothschild Semin Arthritis Rheum 1982, 12, 11-31). Cell proliferation is also a feature of the arthritic joint. Systemic antiproliferative drugs (for example methotrexate) are used in the therapy of rheumatoid arthritis but are limited by side effects. Psoriatic lesions are also characterized by cell proliferation and hypoxia (Dvorak Int Arch Allergy Immunol. 1995, 107, 233-5. Hypoxia drives proliferation of endothelial cells in the retina in diabetic retinopathy and in the choroid of the eye in wet age-related macular degeneration (Das, Prog Retin Eye Res 2003, 22, 721-48).

A number of hypoxic trigger moieties have been disclosed including nitrobenzenes, nitronaphthalenes, nitroimidazoles, nitrofurans, nitrothiophenes, nitropyrroles, nitropyrazoles, benzoquinones, naphthoquinones, indoloquinones and azidobenzenes (for some examples see Naylor, Mini Rev. Med. Chem. 2001 1, 17-29; Tercel, J. Med. Chem. 2001, 44, 3511-3522 and Damen, Bioorg. Med. Chem. 2002, 10, 71-77).

A number of effector moieties have been utilised in the art including nitrogen mustards, phosphoramide mustards, taxanes, enediynes and indole derivatives (for some examples see Naylor, loc cit and Papot, Curr. Med. Chem. Anti Cancer Agents 2002, 2, 155-185).

Hypoxic triggers joined to effectors via a linking group have been described wherein the linking group consists of a carbonate or carbamate for some examples see Naylor, loc cit and Papot loc cit). In these cases it is intended that the intermediate carbonic acid or carbamic acid, formed by the initial hypoxia-driven fragmentation, further fragments to give the active agent.

The combretastatins are a series of stilbene compounds that have powerful antiproliferative activity against cancer cell lines in vitro. Examples of such compounds include combretastatin A4 (U.S. Pat. No. 4,996,237) and combretastatins A1 and A3 (U.S. Pat. No. 5,569,786). Cushman (U.S. Pat. No. 5,430,062) has disclosed a series of Z-stilbenes that are related to the combretastatins and that have anticancer activity. Davis (WO 01 12579) has also disclosed novel stilbenes related to the combretastatins as has Hadfield (WO 02 050007). Stilbene analogues containing an amino group have also been disclosed (U.S. Pat. No. 5,731,353).

It is thought that this antiproliferative activity of these Z-stilbenes is due to an antimitotic action brought about by the inhibition of tubulin polymerisation (see for example Woods et al. Br J Cancer 71, 705-711, 1995). This antimitotic activity requires prolonged exposure of the cells to the compounds. Some of these compounds also have activity against tumour vasculature in vivo, which is distinct from the antimitotic activity observed in vitro. It is thought that the antimitotic activity of these compounds is generally not expressed in vivo because, unlike the antivascular activity, a prolonged exposure of the tumour to the compounds is required and the short elimination half-lives of the compounds preclude this exposure at non-toxic doses. Combretastatin analogues delivered to the tumour by a hypoxia-driven fragmentation strategy offer the potential to deliver prolonged tumour exposure that may be antimitotic while minimising host toxicity. These compounds could also result in generation of active combretastatin compounds at the site of inflammation in rheumatoid arthritis and psoriasis, reducing the cell proliferation that is characteristic of these diseases. Such compounds could also reduce the proliferation of endothelial cells in the retina or choroid associated with diseases such as diabetic retinopathy and wet age-related macular degeneration.

It is an object of this invention to provide prodrugs that on bioreductive activation break down to release an antimitotic stilbene compound.

Thus according to one aspect of the invention we provide a compound of formula (1), or a pharmaceutically acceptable salt thereof: 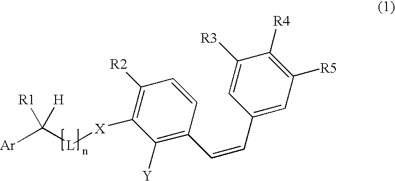
wherein:
Ar is a substituted heteroaryl group bearing at least one nitro or azido group or is a group of formula (2) or (3); 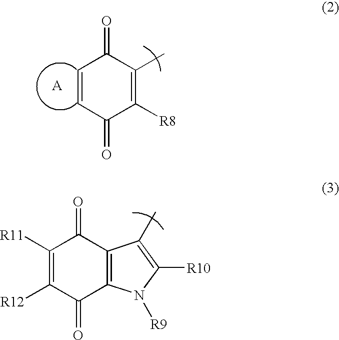
R₁ is hydrogen, optionally substituted alkyl, optionally substituted aryl or optionally substituted heteroaryl;
R₂ is alkyl, alkoxy, thioalkoxy or halo;
R₃, R₄ and R₅ are each independently alkyl, alkoxy, thioalkoxy or halo with—the proviso that at least two of R₃, R₄ or R₅ are alkoxy;
L is —OC(O)— or —OP(O)(OR₆)—;
n is 0 or 1;
X is O, S or NR₇;
Y is hydrogen, alkyl, alkoxy, thioalkoxy, halo, hydroxy or dihydrogenphosphate;
R₆ is hydrogen or alkyl;
R₇ is hydrogen or alkyl;
R₈ is hydrogen, alkoxy or dialkylaminoalkyl;
R₉ is optionally substituted alkyl;
R₁₀ is hydrogen, alkyl, alkoxy or dialkylaminoalkyl;
R₁₁ and R₁₂ are independently hydrogen, alkyl, alkoxy, thioalkoxy, amino, alkylamino, dialkylamino, morpholino, alkylmorpholino, piperidino, alkylpiperidino, piperazino, alkylpiperazino or 1-aziridinyl; and
A, together with the carbon atoms to which it is fused, is an optionally substituted aryl or heteroaryl ring.

As used herein the term “alkyl”, alone or in combinations, means a straight or branched-chain alkyl group containing from one to seven, preferably a maximum of four, carbon atoms such as methyl, ethyl, propyl, isopropyl, butyl, sec-butyl, t-butyl and pentyl. Typically, alkyl group or moiety is a linear or branched alkyl group or moiety containing from 1 to 6 carbon atoms, such as a C₁-C₄ or C₁-C₂ alkyl group or moiety.

As used herein, alkoxy is a said alkyl group, for example a C₁-C₄ or C₁-C₂ alkyl group, which is attached to an oxygen atom.

As used herein, a thioalkoxy group is a said alkyl group which is attached to a sulphur atom.

Optional substituents which may be present on alkyl groups include one or more substituents selected from halogen, ammo, monoalkylamino, dialkylamino, hydroxy, alkoxy, alkylthio, alkylsulphonyl, aryl, heteroaryl, acylamino, alkoxycarbonylamino, alkanoyl, acyloxy, carboxy, sulphate or phosphate groups. A further example of an optional substituent which may be present on alkyl groups is a heterocycloalkyl group. Preferably, the substituents on an alkyl group are selected from halogen, amino, mono(C₁-C₄ alkyl)amino, di(C₁-C₄ alkyl)amino, hydroxy, C₁-C₄ alkoxy and C₁-C₄ alkylthio groups. Typically, alkyl groups are unsubstituted or substituted by one, two or three substitutents. Typically, said substituents which may be present on alkyl groups are themselves unsubstituted. More preferably, an alkyl group is unsubstituted or substituted by 1, 2 or 3 halogen atoms.

The term “halogen” means fluorine, chlorine, bromine or iodine. As used herein, the term “halo” refers to a fluoro, chloro, bromo or iodo substituent. Halo is typically fluoro or chloro.

The term aryl means an unsubstituted phenyl group or a phenyl group carrying one or more, preferably one to three, substituents examples of which are halogen, optionally substituted alkyl, hydroxy, nitro, azido, cyano, amino and alkoxy. Preferably, an aryl group is an unsubstituted phenyl group or a phenyl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, an aryl group is a phenyl group which is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

As used herein, a haloalkyl or haloalkoxy group is a said alkyl or alkoxy group, substituted by one or more said halogen atoms. Typically, a haloalkyl or haloalkoxy group is substituted by 1, 2 or 3 said halogen atoms. Preferred haloalkyl and haloalkoxy groups include perhaloalkyl and perhaloalkoxy groups such as —CZ₃ and —OCZ₃ wherein Z is said halogen atom, for example chlorine or fluorine. Particularly preferred haloalkyl groups are —CF₃ and —CCl₃. Particularly preferred haloalkoxy groups are —OCF₃ and —OCCl₃.

The term heteroaryl is defined herein as a mono- or fused bi-cyclic aromatic group containing one to four heteroatoms selected in any combination from N, S or O atoms. A heteroaryl group is typically a 5- to 10-membered ring, such as a 5- or 6-membered ring, containing at least one heteroatom, for example 1, 2, or 3 heteroatoms chosen from N, S or O atoms. Examples of heteroaryl groups include pyridyl, pyrimidyl, furyl, thienyl, pyrrolyl, pyrazolyl, indolyl, benzofuryl, benzothienyl, benzothiazolyl, oxazolyl, isoxazolyl, thiazolyl, isothiazolyl, imidazolyl, triazolyl, quinolyl and isoquinolyl groups. A heteroaryl group can carry one or more, preferably one to three, substituents examples of which are halogen, optionally substituted alkyl, hydroxy, nitro, azido, cyano, amino and alkoxy. Preferably, a heteroaryl group is an unsubstituted hetoraryl group or a heteroaryl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy substituents. More preferably, a heteroaryl group is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

A heterocycloalkyl ring is typically a non-aromatic, saturated or unsaturated C₃₋₁₀ carbocyclic ring in which one or more, for example, 1, 2 or 3, of the carbon atoms are replaced by a heteroatom selected from N, O or S. Saturated heterocycloalkyl groups are preferred. The term heterocycloalkyl ring includes heterocycloalkyl groups containing 3-6 carbon atoms and one or two oxygen, sulphur or nitrogen atoms. Particular examples of such groups include azetidinyl, pyrrolidinyl, piperidinyl, homopiperidinyl, piperazinyl, homopiperazinyl, morpholinyl or thiomorpholinyl groups.

Substituents which may be present on a heterocycloalkyl ring include one or more groups selected from optionally substituted alkyl, halogen, oxo, hydroxy, alkoxy, alkylthio, amino, alkylamino, dialkylamino, carboxy, alkoxycarbonyl, aminocarbonyl, alkylaminocarbonyl, dialkylaminocarbonyl, alkylsulphonyl, aminosulphonyl, acylamino, alkoxycarbonylamino, alkanoyl, acyloxy, sulphate, phosphate and alkylphosphate. Preferably, a heterocycloalkyl ring is an unsubstituted heterocycloalkyl group or a heterocycloalkyl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, a heterocycloalkyl ring is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

Typically, in the compound of formula (1), R₁ is hydrogen, unsubstituted C₁-C₆ alkyl, a phenyl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy or a heteroaryl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy substituents.

Preferably, in the compound of formula (1), R₁ is hydrogen, unsubstituted C₁-C₄ alkyl, a phenyl group which is unsubstituted or substituted with 1, 2 or 3 unsubstituted substituents selected from halogen, C₁-C₄ alkyl, hydroxy, amino, C₁-C₂ haloalkyl, C₁-C₂ alkoxy and C₁-C₂ haloalkoxy.

A particularly useful group of compounds are those of formula (1) in which R₁ is an alkyl group.

More preferably, in the compound of formula (1), R₁ is hydrogen or unsubstituted C₁-C₂ alkyl.

Typically, in the compound of formula (I), R₂ is unsubstituted C₁-C₆ alkyl, unsubstituted C₁-C₄ alkoxy, unsubstituted thio(C₁-C₄ alkoxy) or a halo group.

More typically, in the compound of formula (1), R₂ is unsubstituted C₁-C₄ alkyl, unsubstituted C₁-C₂ alkoxy, or a chloro or fluoro group.

Preferably, in the compound of formula (1), R₂ is an unsubstituted C₁-C₂ alkoxy group.

Typically, in the compound of formula (1), R₃, R₄ and R₅ are the same or different and each represent unsubstituted C₁-C₆ alkyl, unsubstituted C₁-C₄ alkoxy, unsubstituted thio(C₁-C₄ alkoxy) or a halo group provided that at least two of R₃, R₄ and R₅ are alkoxy.

More typically, in the compound of formula (1), R₃, R₄ and R₅ are the same or different and each represent unsubstituted C₁-C₆ alkyl, unsubstituted C₁-C₂ alkoxy, or a halo group provided that at least two of R₃, R₄ and R₅ are alkoxy.

Preferably, in the compound of formula (1) R₃, R₄ and R₅ are the same or different and each represent unsubstituted C₁-C₂ alkoxy.

Typically, in the compound of formula (1), X is O, S or NR₇, wherein R₇ is hydrogen or unsubstituted C₁-C₆ alkyl, for example unsubstituted C₁-C₄ alkyl.

More typically, in the compound of formula (1), X is O, S or NH.

Preferably, in the compound of formula (1), X is O.

Typically, in the compound of formula (1), n is 0.

Typically, in the compound of formula (1), L is —OC(O)— or —OP(O)(OR₆)—, wherein R₆ is hydrogen or unsubstituted C₁₋₆ alkyl. Typically, in the compound of formula (1), R₆ is hydrogen or unsubstituted C₁₋₄ alkyl.

Preferably, in the compound of formula (1), L is —OC(O)—.

Typically, in the compound of formula (1), Y is selected from hydrogen, unsubstituted C₁-C₆ alkyl, unsubstituted C₁-C₄ alkoxy, unsubstituted thio(C₁-C₄ alkoxy), halo, hydroxy or dihydrogenphosphate substituents.

More typically, in the compound of formula (1), Y is selected from hydrogen, unsubstituted C₁-C₄ alkyl, unsubstituted C₁-C₂ alkoxy or hydroxy substituents.

Preferably, in the compound of formula (1), Y is hydrogen.

Typically, in the compound of formula (1), R₅ is a hydrogen, unsubstituted C₁-C₄ alkoxy or unsubstituted di(C₁-C₆ alkyl)amino(C₁-C₆ alkyl) substituent. More typically, in the compound of formula (1), R₈ is a hydrogen or unsubstituted C₁-C₂ alkoxy substituent.

Typically, in the compound of formula (1), A is a phenyl group or a 5 or 6 membered heteroaryl ring. Typically, the phenyl group or heteroaryl ring is unsubstituted or substituted with 1, 2 or 3 unsubstituted substituents selected from halogen, C₁-C₄ alkyl, hydroxy, amino, C₁-C₄ haloalkyl, C₁-C₄ alkoxy and C₁-C₄ haloalkoxy substituents. Preferably, the phenyl group or heteroaryl ring is unsubstituted or substituted with 1 or 2 unsubstituted substituents selected from halogen, C₁-C₂ alkyl and C₁-C₂ haloalkyl substituents.

Typically, in the compound of formula (1), Ar is a substituted aryl or 5- to 10-membered heteroaryl group bearing at least one nitro or azido group or a group of formula (3).

Typically, when Ar is a substituted aryl or 5- to 10-membered heteroaryl group bearing at least one nitro or azido group, it carries one substituent selected from a nitro or azido group and 0, 1 or 2 further unsubstituted substituents chosen from halogen, C₁-C₆ alkyl, hydroxy, amino, C₁-C₄ haloalkyl, C₁-C₄ alkoxy and C₁-C₄ haloalkoxy substituents. Preferably, said further substituents are chosen from halogen, unsubstituted C₁-C₄ alkyl, hydroxy and amino substituents. More preferably, said substituents are unsubstituted C₁-C₂ alkyl substituents. Typically, when Ar is a substituted aryl or 5- to 10-membered heteroaryl group bearing at least one nitro or azido group, it is a phenyl or a 5- to 6-membered heteroaryl group carrying one substituent selected from a nitro or azido group, and 0, 1 or 2 said further substituents More preferably, when Ar is a substituted aryl or 5- to 10-membered heteroaryl group bearing at least one nitro or azido substituent, said group carries only one substituent which substituent is chosen from a nitro or azido group. Preferably, said substituent is a nitro group.

More typically, when Ar is a substituted aryl or 5- to 10-membered heteroaryl group bearing at least one nitro or azido group, Ar is phenyl or a 5- or 6-membered heteroaryl group, for example a furanyl, imidazolyl or thienyl group, substituted by only one substituent which substituent is a nitro substituent. Particularly useful values of the moiety Ar include nitroimidazole groups, for example 2-nitroimidazol-5-yl and nitrothiophene groups, for example 5-nitrothien-2-yl. Further particularly useful examples of the moiety Ar include nitrofuranyl groups, for example 5-nitrofuran-2-yl.

Typically, when Ar is a group of formula (3), R₉ is an unsubstituted C₁-C₆ alkyl group. More typically, R₉ is unsubstituted C₁-C₄ alkyl group. Preferably, R₉ is an unsubstituted C₁-C₂ alkyl group.

Typically, when Ar is a group of formula (3), R₁₀ is selected from hydrogen, unsubstituted C₁-C₆ alkyl, unsubstituted C₁-C₄ alkoxy or unsubstituted di(C₁-C₆ alkyl)amino(C₁-C₆ alkyl) substituents. More typically, R₁₀ is selected from hydrogen, unsubstituted C₁-C₄ alkyl or unsubstituted C₁-C₂ alkoxy substituents. Preferably, R₁₀ is an unsubstituted C₁-C₂ alkyl group.

Typically, when Ar is a group of formula (3), R₁₁ and R₁₂ are the same or different and each represent a unsubstituted substituent selected from hydrogen, C₁-C₆ alkyl, C₁-C₄ alkoxy, thio(C₁-C₄ alkoxy), amino, (C₁-C₆ alkyl)amino, di(C₁-C₆ alkyl)amino, morpholino, (C₁-C₆ alkyl)morpholino, piperidino, (C₁-C₆ alkyl)piperidino, piperazino, (C₁-C₆ alkyl)piperazino and 1-aziridinyl substituents. More typically, R₁₁ and R₁₂ are the same or different and each represent an unsubstituted substituent selected from hydrogen, C₁-C₄ alkyl, C₁-C₂ alkoxy, thio(C₁-C₂ alkoxy) and (C₁-C₄ alkyl)piperidino substituents. Preferably, R₁₁ and R₁₂ are the same or different and each represent a substituent selected from hydrogen, unsubstituted C₁-C₂ alkoxy and unsubstituted (C₁-C₂ alkyl)piperidino substituents.

In the compound of formula (1), preferably R₂, R₃, R₄ and R₅ are the same and represent unsubstituted methoxy. It is further preferred that, in the compound of formula (1), R₂, R₃, R₄ and R₅ are the same and represent unsubstituted methoxy and Y represents hydrogen.

Preferably, in the compound of formula (1),
R₁ is hydrogen, unsubstituted C₁-C₆ alkyl, a phenyl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy or a heteroaryl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy;
R₂ is unsubstituted C₁-C₆ alkyl, unsubstituted C₁-C₄ alkoxy, unsubstituted thio(C₁-C₄ alkoxy) or a halo group;
R₃, R₄ and R₅ are the same or different and each represent unsubstituted C₁-C₆ alkyl, unsubstituted C₁-C₄ alkoxy, unsubstituted thio(C₁-C₄ alkoxy) or a halo group provided that at least two of R₃, R₄ and R₅ are alkoxy;
n is 0 or 1, wherein when n is 1, L is —OC(O)— or —OP(O)(OR₆)—;
R₆ is hydrogen or unsubstituted C₁₋₆ alkyl;
X is O, S or NR₇;
R₇ is hydrogen or unsubstituted C₁-C₆ alkyl;
Y is selected from hydrogen, unsubstituted C₁-C₄ alkyl, unsubstituted C₁-C₂ alkoxy or hydroxy substituents; and
Ar is either (a) a substituted aryl or 5 to 10 membered heteroaryl group which carries one substituent selected from a nitro or azido group and 0, 1 or 2 further unsubstituted substituents chosen from halogen, C₁-C₆ alkyl, hydroxy, amino, C₁-C₄ haloalkyl, C₁-C₄ alkoxy and C₁-C₄ haloalkoxy substituents; or (b) a group of formula (3), wherein:
   R₉ is an unsubstituted C₁-C₆ alkyl group;
   R₁₀ is selected from hydrogen, unsubstituted C₁-C₆ alkyl, unsubstituted C₁-C₄ alkoxy or unsubstituted di(C₁-C₆ alkyl)amino(C₁-C₆alkyl) substituents; and
   R₁₁ and R₁₂ are the same or different and each represent an unsubstituted substituent selected from hydrogen, C₁-C₆ alkyl, C₁-C₄ alkoxy, thio(C₁-C₄ alkoxy), amino, (C₁-C₆ alkyl)amino, di(C₁-C₆ alkyl)amino, morpholino, (C₁-C₆ alkyl)morpholino, piperidino, (C₁-C₆ alkyl)piperidino, piperazino, (C₁-C₆ alkyl)piperazino and 1-aziridinyl substituents.
      More preferably, in the compound of formula (1),
R₁ is hydrogen or unsubstituted C₁-C₂ alkyl;
R₂ is an unsubstituted C₁-C₂ alkoxy group;
R₃, R₄ and R₅ are the same or different and each represent unsubstituted C₁-C₂ alkoxy;
n is 0 or 1, wherein when n is 1, L is —OC(O)—;
X is O;
Y is hydrogen; and
Ar is either (a) 2-nitroimidazol-5-yl, 5-nitrothien-2-yl or 5-nitrofuran-2-yl; or (b) a group of formula (3), wherein:
   R₉ is an unsubstituted C₁-C₂ alkyl group;
   R₁₀ is an unsubstituted C₁-C₂ alkyl group; and
   R₁₁ and R₁₂ are the same or different and each represent a substituent selected from hydrogen, unsubstituted C₁-C₂ alkoxy and unsubstituted (C₁-C₂ alkyl)piperidino substituents.

More preferably, the compound of formula (1) is selected from 1-(4-Methoxy-3-(5-nitrothien-2-yl)methoxy)phenyl-2-(3,4,5-trimethoxy)phenyl-Z-ethene, 1-(4-Methoxy-3-(1-(5-nitrothien-2-yl)ethoxy))phenyl-2-(3,4,5-trimethoxy)phenyl-Z ethene, 1-(4-Methoxy-3-(5-nitrothien-2-yl)methoxycarbonyloxy)phenyl-2-(3,4,5-trimethoxy)phenyl-Z-ethene, 5-Methoxy-3-((3,4,4′,5-tetramethoxy-(Z)-stilbene-3′-yl)oxy)methyl-1,2-dimethylindole-4,7-dione and 3-((3,4,4′,5-Tetramethoxy-(Z)-stilbene-3′-yl)oxy)methyl-1,2-dimethyl-5-(4-methylpiperazin-1-yl)indole-4,7-dione.

Where one or more functional groups in compounds of formula (1) are sufficiently basic or acidic the formation of salts is possible. Suitable salts include pharmaceutically acceptable salts for example acid addition salts including hydrochlorides, hrdrobromides, phosphates, sulphates, hydrogen sulphates, alkylsulphonates, arylsulphonates, acetates, benzoates, citrates, maleates, fumarates, succinates, lactates and tartrates, salts derived from inorganic bases including alkali metal salts such as sodium or potassium salts, alkaline earth metal salts such as magnesium or calcium salts, and salts derived from organic amines such as morpholine, piperidine or dimethylamine salts.

Those skilled in the art will recognise that compounds of formula (1) may exist as stereoisomers and/or geometrical isomers and accordingly the present invention includes all such isomers which have anticancer activity and mixtures thereof.

It is a further object of this invention to provide methods for the preparation of compounds of Formula (1).

Compounds of Formula (1) may be prepared by a number of processes as generally described below and more specifically in the Examples hereinafter. In the following process description, the symbols Ar, R₁ and R₂ when used in the formulae depicted are to be understood to represent those groups described above in relation to Formula (1) unless otherwise indicated. In the schemes described below it may be necessary to employ protecting groups that are then removed during the final stages of the synthesis. The appropriate use of such protecting groups and processes for their removal will be readily apparent to those skilled in the art.

Compounds of Formula (1) in which X is O or S and n is 0 can be prepared by Mitsunobu reaction of an alcohol of formula (4) with a stilbene of formula (5) in a solvent such as an ether solvent, for example tetrahydrofuran, diethyl ether or dioxan or in a solvent such as an aromatic hydrocarbon for example benzene or toluene or in a solvent such as an aprotic solvent for example dimethylformamide, in the presence of a phosphine for example triphenylphosphine or tri-n-butylphosphine and in the presence of an azo compound such as diethylazodicarboxylate, diisopropylazodicarboxylate or 1,1′-(azodicarbonyl)dipiperidine at a temperature from about 0° C. to about the reflux temperature of the solvent, conveniently at room temperature.

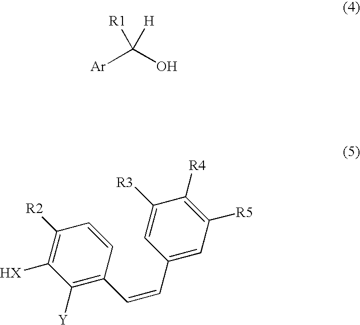

Alcohols of formula (4) are either known or can be prepared by standard methods apparent to one skilled in the art. Such methods include treatment of an aldehyde or ketone of formula (6) with a reducing agent, for example a borohydride reducing agent such as sodium borohydride in a solvent such as an alcoholic solvent for example methanol at a temperature between about −20° C. to room temperature, preferably around 0° C. Such methods also include the treatment of an aldehyde of formula (7) with an organometallic compound of formula (8) in which M represents a metal, metal halide or dialkylmetal, for example, Li, ZnBr, Mglr or MgI or dialkylaluminium in a solvent such as an ether solvent, for example tetrahydrofuran or diethyl ether or in an aromatic solvent for example benzene or toluene at a temperature of between about −78° C. to about the reflux temperature of the solvent, preferably from about 0° C. to room temperature. Where Ar is a substituted aryl or heteroaryl group bearing at least one nitro group such methods also include the aromatic electrophilic nitration of the appropriate aryl substrate with an appropriate nitrating agent at a temperature of between about −78° C. and room temperature. Appropriate nitrating agents are, for example, nitric acid in a solvent such as an acid anhydride for example acetic anhydride or in a solvent such as an acid for example sulphuric acid or acetic acid; nitronium tetrafluoroborate in a solvent such as an ether solvent, for example tetrahydrofuran or diethyl ether or in a solvent such as acetonitrile or glacial acetic acid or in a solvent such as a chlorinated solvent for example dichloromethane or dinitrogen tetroxide in a solvent such as an ether solvent, for example tetrahydrofuran or diethyl ether or in a solvent such as acetonitrile or glacial acetic acid or in a solvent such as a chlorinated solvent for example dichloromethane or in an aromatic solvent for example benzene or toluene.

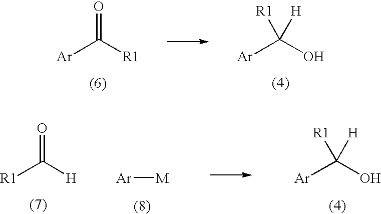

Compounds of formula (1) in which n=0 can also be prepared by treatment of a halide of formula (9), in which Hal represents a chlorine, bromine or iodine atom, with a compound of formula (5), in a solvent such as an aprotic solvent such as dimethylformamide or in an ether solvent such as diethyl ether or tetrahydrofuran, or in a ketone solvent such as acetone in the presence of a base such as a metal carbonate for example potassium carbonate or silver(I)carbonate or a base such as a metal hydride for example sodium hydride or potassium hydride, at a temperature of between about −78° C. to about the reflux temperature of the solvent preferably between 0° and room temperature.

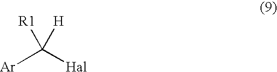

Halides of formula (9) are either known or can be prepared by standard methods apparent to one skilled in the art. Such methods include the halogenation of a compound of formula (10) with a halogenating agent such as N-bromosuccinimide, N-chlorosuccinimide or bromine in a solvent such as a chlorinated solvent for example dichloromethane or carbon tetrachloride at a temperature of about between about 0° C. and the reflux temperature of the solvent.

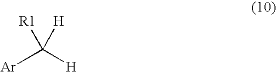

Compounds of Formula (1) in which X is O, n is 1 and L is —OC(O)— can be prepared by treatment of an alcohol of formula (4) with an acid chloride of formula (11) in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

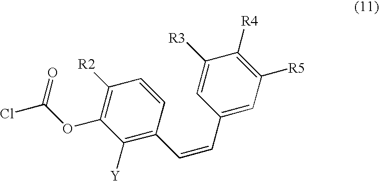

Acid chlorides of formula (11) are either known or can be prepared by standard methods apparent to one skilled in the art. Such methods include treatment of a compound of formula (5) in which X═O with phosgene or triphosgene in a solvent such as a chlorinated solvent for example dichloromethane or trichloromethane with or without the addition of dimethylformamide at a temperature of around 0° C. to room temperature.

Compounds of Formula (1) in which X is NH, n is 1 and L is —OC(O)— can be prepared by treatment of an alcohol of formula (4) with an isocyanate of formula (12) in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

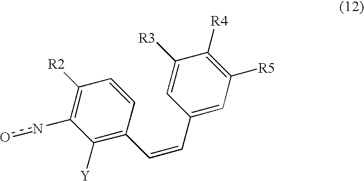

Compounds of formula (1) in which X is NR₇, n is 1 and L is —OC(O)— can be prepared by treatment of a chloroformate of formula 13 with a compound of the formula (5) in which X═NR₇ in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

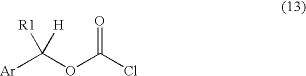

Compounds of formula (1) in which n is 1 and L is —OP(O)(OR₆)— can be prepared by treatment of an alcohol of formula (4) with a compound of the formula (14) in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

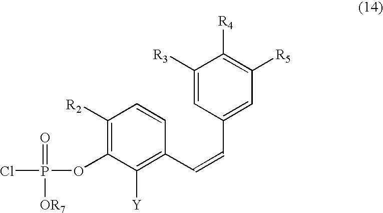

Compounds of formula (1), wherein n is 1, L is —OC(O)— and X is S can, of course be made by the reaction of an appropriate acid chloride of formula Ar—CHR₁—O—C(O)Cl with a thiol having the formula (15), in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

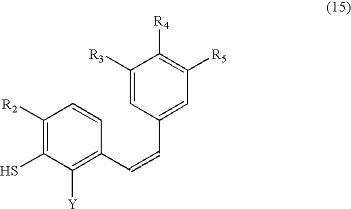

Compounds of formula (1) can also be synthesized from other compounds of formula (1) by the application of standard methods, including substitution reactions, functional group transformations, bond-forming reactions and cyclisations known in the art.

The starting materials for the above described schemes are commercially available or can be synthesized using standard techniques.

Preparation of a compound of Formula (1) as a single enantiomer or, where appropriate, diastereomer may be effected by synthesis from an enantiomerically pure starting material or intermediate or by resolution of the final product in a conventional manner.

The compounds of the invention may be administered as a sole therapy or in combination with other treatments. For the treatment of solid tumours compounds of the invention may be administered in combination with radiotherapy or in combination with other anti-tumour substances for example those selected from mitotic inhibitors, for example vinblastine, vincristine, vinorelbine, paclitaxel and docetaxel; alkylating agents, for example cisplatin, carboplatin, oxaliplatin, nitrogen mustard, melphalan, chlorambucil, busulphan and cyclophosphamide; antimetabolites, for example 5-fluorouracil, cytosine arabinoside, gemcitabine, capecitabine, methotrexate and hydroxyurea; intercalating agents for example adriamycin and bleomycin; enzymes, for example aspariginase; topoisomerase inhibitors for example etoposide, teniposide, topotecan and irinotecan; thymidylate synthase inhibitors for example raltitrexed; biological response modifiers for example interferon; antibodies for example edrecolomab and trastuzumab; receptor tyrosine kinase inhibitors for example gefitinib and erlotinib; and anti-hormones for example tamoxifen. Further examples of antibodies which can be administered in combination with the compounds of the invention include, for example bevacizumab and cetuximab. A further example of a receptor tyrosine kinase inhibitor which can be administered in combination with the compounds of the invention is imatinib. Such combination treatment may involve simultaneous or sequential application of the individual components of the treatment.

For the prophylaxis and treatment of disease the compounds according to the invention may be administered as pharmaceutical compositions selected with regard to the intended route of administration and standard pharmaceutical practice. Such pharmaceutical compositions may take a form suitable for oral, buccal, nasal, topical, rectal or parenteral administration and may be prepared in a conventional manner using conventional excipients. For example for oral administration the pharmaceutical compositions may take the form of tablets or capsules. The compositions for oral administration may also be in the form of lozenges, aqueous or oily suspensions, dispersible powders or granules. For nasal administration or administration by inhalation the compounds may be conveniently delivered as a powder or in solution. Topical administration may be as an ointment or cream and rectal administration may be as a suppository. For parenteral injection (including intravenous, subcutaneous, intramuscular, intravascular or infusion) the composition may take the form of, for example, a sterile solution, suspension or emulsion. The compounds of the invention may also be administered as suppositories.

The dose of a compound of the invention required for the prophylaxis or treatment of a particular condition will vary depending on the compound chosen, the route of administration, the form and severity of the condition and whether the compound is to be administered alone or in combination with another drug. Thus the precise dose will be determined by the administering physician but in general daily dosages may be in the range 0.001 to 100 mg/kg preferably 0.1 to 10 mg/kg.

Typically, daily dosage levels are from 0.05 mg to 2 g, for example from 5 mg to 1 g.

The present invention therefore provides a pharmaceutical composition comprising a compound of formula (1), or a pharmaceutically acceptable salt thereof, and a pharmaceutically acceptable carrier or diluent.

A further feature of the present invention is a compound of formula (1), or a pharmaceutically acceptable salt or solvate thereof, for use as a medicament. In particular, the present invention provides a compound of formula (1), or a pharmaceutically acceptable salt thereof, for the treatment of the human or animal body.

The compounds of the present invention are therapeutically useful in treating, preventing, ameliorating or reducing incidence of a proliferative disorder. Typically, the proliferative disorder is a hypoxic disorder. A hypoxic disorder is typically a disorder in which diseased cells are present in a hypoxic environment. Examples of the disorders that can be treated, prevented, ameliorated or disorders whose incidence can be reduced, include cancer, rheumatoid arthritis, psoriatic lesions, diabetic retinopathy or wet age-related macular degeneration.

Typically, the disorder is cancer. Preferably the cancer is a hypoxic cancer. A hypoxic cancer is, of course, a cancer wherein cancerous cells are in a hypoxic environment. Most preferably, the cancer is a solid tumour or leukaemia. Typically the leukaemia is leukaemia involving the spleen or bone marrow.

According to a further aspect of the invention there is provided the use of a compound of formula (1), or a pharmaceutically acceptable salt or solvate thereof, in the manufacture of a medicament for use in the therapy of a warm-blooded animal, for example a human, suffering from a proliferative disease for example cancer. In particular, the present invention provides the use of a compound of formula (1), or a pharmaceutically acceptable salt thereof, in the manufacture of a medicament for use in the treatment of the human or animal body, for the prevention or treatment of a said proliferative disorder.

According to a further aspect of the invention there is provided a compound of formula (1), or a pharmaceutically acceptable salt or solvate thereof, for use in a method of treatment of the human or animal body by therapy. In particular, the present invention provides a method of ameliorating or reducing the incidence of a said proliferative disorder in a patient, which method comprises administering to said patient an effective amount of a compound of formula (1), or a pharmaceutically acceptable salt thereof.

A number of enzymes are capable of reducing aryl and heteroaryl nitro groups. Strategies that increase the activity of such enzymes within solid tumours can therefore increase further the activity of prodrugs dependent on nitro reduction. Similarly a number of enzymes are capable of reducing quinones and indoloquinones and therefore similar strategies are possible to increase the effectiveness of drugs requiring activation by quinone reduction. Such strategies include linking such enzymes to a tumour-targeting antibody, administering such enzyme antibody conjugates to a host with a solid tumour then, after the conjugate has localised to the tumour, administering the prodrug. This approach is known as Antibody Directed Enzyme Prodrug Tharapy (ADEPT). Alternatively the gene encoding for the enzyme might be delivered selectively and/or expressed selectively, in the tumour before administration of the prodrug. This approach is known as Gene Directed Enzyme Prodrug Therapy (GDEPT). When the gene is delivered by a viral vector the approach is sometimes known as Virus Directed Enzyme Prodrug Therapy (VDEPT).

Anlezark has disclosed nitroreductases and their use in an ADEPT strategy. Prodrugs for use in this strategy were also disclosed (U.S. Pat. No. 5,633,158 and U.S. Pat. No. 5,977,065). In WO 00 047725 Anlezark provides further disclosures of nitroreductase enzymes and their use in GDEPT strategies. Denny (WO 00 064864) has disclosed nitroaryl and nitroheteroaryl prodrugs for use in a GDEPT strategy. The use of quinone-reducing enzymes in ADEPT, GDEPT and MDEPT (Macromolecule Directed Enzyme Prodrug Therapy) is discussed in Skelly et al. Mini Rev Med Chem. 2001, 1, 293-306.

Thus it is a further object of this invention to provide the use of compounds of formula (1) in combination with a reductase, an antibody-reductase conjugate, a macromolecule-reductase conjugate or DNA encoding a reductase gene, in a method of treatment for the human body. Thus, the present invention provides a method of ameliorating or reducing the incidence of a said proliferative disorder in a patient, which method comprises administering to said patient an effective amount of
(a) a compound of formula (1), or a pharmaceutically acceptable salt thereof; and
(b) a reductase, an anti-body reductase conjugate, a macromolecule-reductase conjugate or DNA encoding a reductase gene.

Further, the present invention provides a product containing
(a) a compound of formula (1), or a pharmaceutically acceptable salt thereof; and
(b) a reductase, an anti-body reductase conjugate, a macromolecule-reductase conjugate or DNA encoding a reductase gene
   for simulataneous, separate or sequential use in the treatment of a proliferative condition.

In a further aspect, the present invention provides a compound of formula (1′), or a pharmaceutically acceptable salt thereof: 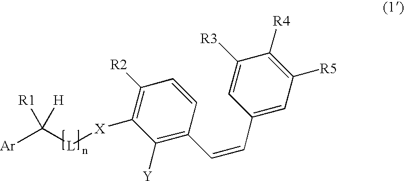
wherein:
Ar is a substituted heteroaryl group bearing at least one nitro or azido group or is a group of formula (2′) or (3′) 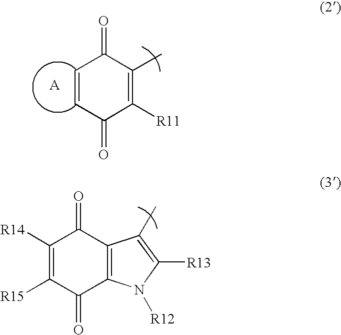
R₁ is hydrogen, optionally substituted alkyl, optionally substituted alkenyl, optionally substituted alkynyl, aryl or COR₆;
R₂ is alkyl, alkoxy, thioalkoxy or halo;
R₃, R₄ and R₅ are each independently alkyl, alkoxy, thioalkoxy or halo with the proviso that at least two of R₃, R₄ or R₅ are alkoxy;
L is —OC(O)— or —OP(O)(OR₇)—;
n is 0 or 1;
X is O, S or NR₈;
Y is hydrogen, alkyl, alkoxy, thioalkoxy, halo, hydroxy or dihydrogenphosphate;
R₆ is OR₈ or NR₉R₁₀;
R₇, R₈, R₉ and R₁₀ are each independently hydrogen or alkyl;
R₁₁ is hydrogen, alkoxy or dialkylaminoalkyl;
R₁₂ is optionally substituted alkyl;
R₁₃ is hydrogen, alkyl, alkoxy or dialkylaminoalkyl;
R₁₄ and R₁₅ are independently hydrogen, alkyl, alkoxy, thioalkoxy, amino, alkylamino, dialkylamino morpholino, alkylmorpholino, piperidino, alkylpiperidino, piperazino, alkylpiperazino or 1-aziridinyl; and
A is an optionally substituted aryl or heteroaryl ring.

In a compound of formula (1′), an alkenyl group may be for example an olefinic group containing from two to seven carbon atoms, for example ethenyl, n-propenyl, i-propenyl, n-butyenyl, i-butenyl, s-butenyl and t-butenyl.

As used herein, in a compound of formula (1′), an alkynyl group may be for example an ethynyl, propynyl or butynyl group.

In a compound of formula (1′), optional substituents which may be present on alkenyl or alkynyl groups are the same as those which may be present on alkyl groups, as described above.

In the compound of formula (1′), particularly useful values of the moiety Ar include nitroimidazole groups, for example 2-nitroimidazol-5-yl, and nitrothiophene groups, for example 5-nitro-thiophen-2-yl.

Where one or more functional groups in compounds of formula (1′) are sufficiently basic or acidic the formation of salts is possible. Suitable salts include those as described above with respect to a compound of formula (1).

Those skilled in the art will recognise that compounds of formula (1′) may exist as stereoisomers and/or geometrical isomers and accordingly the present invention includes all such isomers which have anticancer activity and mixtures thereof.

The present invention therefore provides a pharmaceutical composition comprising a compound of formula (1′), or a pharmaceutically acceptable salt thereof, and a pharmaceutically acceptable carrier or diluent.

A further feature of the present invention is a compound of formula (1′), or a pharmaceutically acceptable salt or solvate thereof, for use as a medicament. In particular, the present invention provides a compound of formula (1′), or a pharmaceutically acceptable salt thereof, for the treatment of the human or animal body.

According to a further aspect of the invention there is provided the use of a compound of formula (1′), or a pharmaceutically acceptable salt or solvate thereof, in the manufacture of a medicament for use in the therapy of a warm-blooded animal, for example a human, suffering from a proliferative disease for example cancer. In particular, the present invention provides the use of a compound of formula (1), or a pharmaceutically acceptable salt thereof, in the manufacture of a medicament for use in the treatment of the human or animal body, for the prevention or treatment of a said proliferative disorder.

The ability of compounds of the invention to release cytotoxic or cytostatic agents selectively under hypoxic conditions can be assessed by using, for example, one or more of the procedures set out below:

Radiolysis

In the hypoxic environments of solid tumours, prodrugs can be reduced by one-electron processes that are inhibited in the normoxic environments of normal tissues.

Radiolysis demonstrates the ability of bioreductively-activated prodrugs to release the active drug after one-electron reduction. Compounds were dissolved in an isopropanol/water mixture (50:50) at a concentration of 50 μM or below. Solutions, in gas-tight syringes, were saturated with nitrous oxide before irradiation in a ⁶⁰Co source at a dose rate of 3.9 Gy min⁻¹ (as determined by Fricke dosimetry: H. Fricke and E. J. Hart, “Chemical Dosimetry” in Radiation Dosimetry Vol. 2 (F. H. Attrix and W. C. Roesch. Eds.), pp 167-239. Academic Press New York, 1966.). Solutions were analysed for released drug by HPLC. The radiation chemical yields (G-values) of combretastatin A4 release obtained in this assay for selected example compounds are shown in Table 1.

TABLE 1
Radiation chemical yields from steady state radiolysis

| Compound of Example No. | G (μmoles · J⁻¹) |
|---|---|
| 1 | 0.23 |
| 2 | 0.33 |
| 4 | 0.44 |
| 5 | 0.34 |

Metabolism in Tumour Homogenates
Useful bioreductive prodrugs can be shown to release the active drug selectively under conditions of low oxygen in the presence of tumour homogenate in this assay. Freshly-excised CANT tumours (approximately 0.5 to 1 g) were homogenised in 15 ml of ice-cold 50 mmol dm 3 potassium phosphate buffer at pH 7.4. The homogenates were centrifuged at 1000 RPM for 10 min and the supernatants stored on ice. The metabolism of 5 μmol dm⁻³ prodrug in air and N₂ was performed with 0.5 ml tumour homogenate (˜3 mg of protein by Bradford assay) with 100 μmol dm⁻³ NADPH in 50 mmol dm⁻³ potassium phosphate buffer at pH 7.4 incubated at 37° C. Samples (60 μl) were taken at regular intervals and added to an equivalent volume of acetonitrile, then mixed and centrifuged at 14, 300 RPM for 2 min prior to product analysis by HPLC. In this test the compound of Example 1 produced combretastatin A4 at a rate of 310 pmol. min⁻¹ .mg protein⁻¹ under nitrogen but only 30 nmol. min⁻¹ .mg protein⁻¹ under air.
Cellular Cytotoxicity
The cytotoxic or cytostatic properties of compounds of formula (1) and compounds of formula DrXH can be assessed for example, by use, for example, of this assay. The Celltiter 96® Aqᵤₑₒᵤₛ One Solution Cell Proliferation Assay kit (Promega Corporation, USA) which is a calorimetric method for determining the number of viable cells in proliferation or cytotoxicity assays was used. In this assay the MTS tetrazolium compound (Owen's Reagent) is bioreduced by viable cells into a coloured formazan product which is soluble in tissue culture medium and can be measured by recording absorbance at 490 nm with a 96 well plate reader. A549 cells were seeded in Eagles Minimum Essential Medium supplemented with 10% foetal calf serum and non-essential amino acids at 10³ cell per well on a 96 well plate and allowed to attach for 24 h. Compounds were dissolved in DMSO and diluted with cell culture medium before addition to The cells were exposed to test compound (0 to 2 μmol dm⁻³) for 6 h then incubated for a further 72 h. The MTS reagent was added to each well, left for 4 h, then the absorbance measured at 490 nm with a 96 well plate reader. 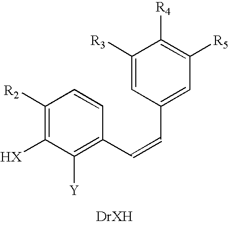
The invention is illustrated by the following non-limiting Examples in which, unless otherwise stated:
DMF means dimethylformamide
THF means tetrahydrofuran
MeOH means methyl alcohol
EtOAc means ethyl acetate
DCM means dichloromethane
TLC means thin-layer chromatography
TFA means trifluoroacetic acid
MeCN means acetonitrile
LC-RT means the retention time given by high-performance liquid chromatography performed using a Waters Integrity system with detection by mass spectroscopy with electron impact ionization. Chromatography used a Hichrom RPB column (100×3.2 mm) with various solvent gradients of either A: 10% acetonitrile, water or B: 5% Acetonitrile, 0.1% TFA with C: Acetonitrile, at a flow rate of 0.5 ml/min.

### EXAMPLE 1

### 1-(4-Methoxy-3-(5-nitrothien-2-yl)methoxy)phenyl-2-(3,4,5-trimethoxy)phenyl-Z-ethene

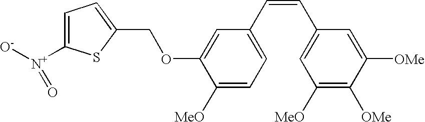

5-Nitro-2-hydroxymethylthiophene (500 mg, 3.14 mmol) was dissolved in THF (5 ml) together with triphenylphosphine (1.68 g, 6.28 mmol) and combretastatin A4 (1.98 g, 6.28 mmol). To this solution was added diethylazodicarboxylate (1.09 g, 6.28 mmol) and the solution heated at 50° C. for 3 h, evaporated to dryness and the residue purified on silica (25% EtOAc/hexane) to give a pale yellow solid (mp 88-90° C., 810 mg, 57%) after recrystallisation from EtOAc/hexane. LC-RT 9.98 minutes (TFA 50-100%; MS (m/z, %) 457 (M+, 85%), 316 (61%), 301 (47%), 252 (100%). Anal. C, 60.5; H, 5.1, N, 2.9% C₂₃H₂₃NO₇S requires C, 60.4; H, 5.1, N, 3.1%.

### EXAMPLE 2

### 1-(4-Methoxy-3-(1-(5-nitrothien-2-yl)ethoxy))phenyl-2-(3,4,5-trimethoxy)phenyl-Z-ethene

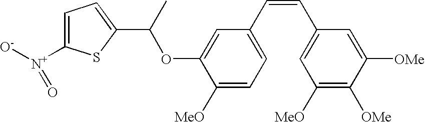

Diethylazodicarboxylate (357 mg, 2.05 mmol) was added dropwise to a solution of alcohol 2-(1-hydroxyethyl)-5-nitrothiophene (55 mg, 0.32 mmol), combretastatin A4 (648 mg, 2.05 mmol), triphenylphosphine (288 mg, 1.10 mmol) and THF (3 mL). The reaction was stirred for 16 hours at ambient temperature and was then partitioned (EtOAc, brine), aqueous phase extracted (EtOAc), organic phase washed (H₂O, brine), dried (NgSO₄) and concentrated in vacuo. Flash chromatography, eluting sequentially with 50% EtOAc/hexane then 100% EtOAc, furnished the desired product as a yellow oil (15 mg, 10%). LC-RT 3.85 minutes (100% MeCN). MS (m/z, %) 471 (M⁺), 425 (M⁺—NO₂), 316, 301, 141.

### EXAMPLE 3

### 1-(4-Methoxy-3-(5-nitrothien-2-yl)methoxycarbonyloxy)phenyl-2-(3,4,5 trimethoxy)phenyl-Z-ethene

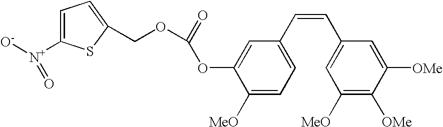

Phosgene (0.5 ml, 1 mmol) was dissolved in DCM (1 ml) at 0° C. and under an argon atmosphere. To this solution was added combretastatin A4 (100 mg, 0.32 mmol) in DCM (0.5 ml) followed by triethylamine (60 μl, 0.4 mmol) after 1 h at 0° C. The solution was then stirred for 18 h while warming to 20° C., and then evaporated to dryness and re-dissolved in DCM (1 ml). To this was added triethylamine (56 ml, 0.4 mmol) and 5-nitro-2-hydroxymethylthiophene (60 mg, 0.376 mmol) and the solution stirred at 20° C. for 5 days then evaporated to dryness. The residue was purified on silica (hexane/EtOAc, 4:1) to give a residue that was then purified by preparative HPLC (CH₃CN/H₂O, 9:1) to give 10 mg (7.5%) of a pale yellow gum. LC-RT 3.22 minutes (100% MeCN). MS (m/z, %) 501 (M+, 100%), 316 (55%), 301 (46%), 252 (50%).

### EXAMPLE 4

### 5-Methoxy-3-((3,4,4′,5-tetramethoxy-(Z)-stilbene-3′-yl)oxy)methyl-1,2-dimethylindole-4,7-dione

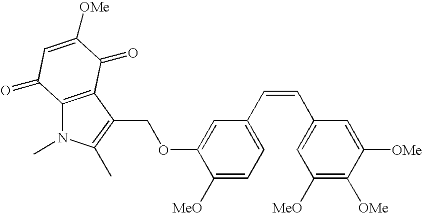

Thionyl chloride (0.78 g, 6.5 mmol) was added drop-wise with stirring to a solution of 3-hydroxymethyl-5-methoxy-1,2-dimethylindole-4,7-dione (0.03 g, 0.13 mmol) in DCM (2 mL). The solution was stirred at 20° C. for 1.5 h and evaporated to dryness. The residue was redissolved in DMF (1.5 mL) and potassium carbonate (0.054 g, 0.39 mmol) added, followed by combretastatin A4 (0.123 g, 0.39 mmol). The solution was stirred at 20° C. for 18 h and EtOAc (50 mL) added. The solution was washed with water (25 ml), saturated sodium bicarbonate (25 ml) and brine (25 ml), dried (MgSO₄) and evaporated. The residue was purified by radial chromatography, eluting with EtOAc to give the title compound (15 mg, 21%) as an orange waxy solid: mp 192-194° C.; LC-RT 5.13 minutes (80% MeCN). MS (m/z, %) 533 (M⁺, 3%), 316 (27%), 301 (24%), 218 (100%). ¹H NMR (60 MHz, CDCl₃) δ 7.02 (m, 2H), 6.79 (m, 2H), 6.44 (m, 3H), 5.57 (s, 1H), 5.12 (s, 2H), 3.85 (s, 3H), 3.78 (s, 6H), 3.67 (s, 6H), 2.26 (s, 3H) ppm. Anal. Fnd. C, 67.1; H, 5.9, N, 2.6% C₃₀H₃₁NO₈ requires C, 67.5; H, 5.9, N, 2.6%.

### EXAMPLE 5

### 3-((3,4,4′,5-Tetramethoxy-(Z)-stilbene-3′-yl)oxy)methyl-1,2-dimethyl-5-(4 methylpiperazin-1-yl)indole-4,7-dione

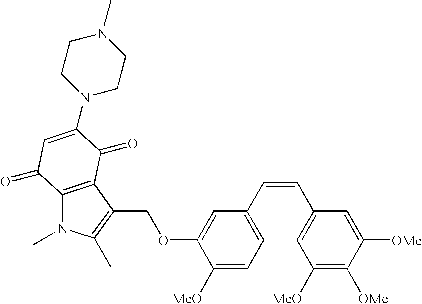

The compound from Example 4 (53 mg, 0.1 mmol) was dissolved in anhydrous DMF (1.5 mL) together with 4-methylpiperazine (0.4 mL, ca 4 mmol). The solution was stirred at room temperature for 48 h and then EtOAc (25 mL) added. The solution was washed with saturated sodium bicarbonate (25 mL) and brine (25 mL), dried (MgSO₄) and evaporated. The residue was purified on silica gel, eluting with 50% MeOH/EtOAc to give the title compound (28 mg, 47%) as a dark red gum. LC-RT 4.45 minutes (TFA 50-100%). MS (m/z, %) 601 (M+, 7%), 316 (26%), 301 (19%), 286 (100%); ¹H NMR (60 MHz, CDCl₃) δ 7.21 (s, 1H), 7.0 (m, 3H), 6.5 (s, 1H), 6.45 (d, J=5 Hz, 2H), 5.44 (s, 1H), 5.06 (s, 2H), 3.84 (s, 3H), 3.78 (s, 6H), 3.68 (s, 6H), 3.41 (m, 4H), 2.54 (m, 4H), 2.34 (s, 3H), 2.23 (s, 3H) ppm. MS (m/z, %) 601 (M⁺, 5%), 316 (19%), 301 (17%), 286 (100%). Anal. Fnd. C, 60.6; H, 6.2, N, 6.1% C₃₄H₃₉N₃O₇.4H₂O requires C, 60.6; H, 7.0, N; 6.2%.

1. A compound of formula (1), or a pharmaceutically acceptable salt thereof: 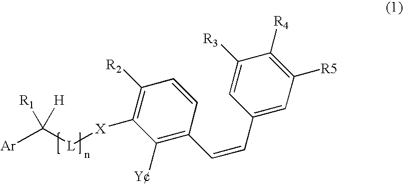
wherein:
Ar is a substituted heteroaryl group bearing at least one nitro or azido group or is a group of formula (2) or (3); 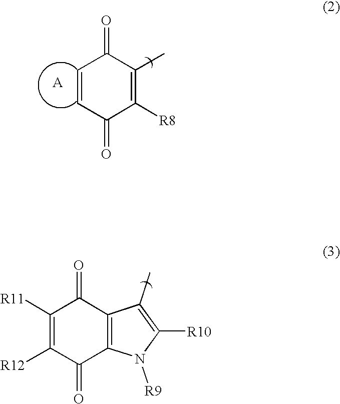
R₁ is hydrogen, optionally substituted alkyl, optionally substituted aryl or optionally substituted heteroaryl;
R₂ is alkyl, alkoxy, thioalkoxy or halo;
R₃, R₄ and R₅ are each independently alkyl, alkoxy, thioalkoxy or halo with
the proviso that at least two of R₃, R₄ or R₅ are alkoxy;
L is —OC(O)— or —OP(O)(OR)—;
n is 0 or 1;
X is O, S or NR₇;
Y is hydrogen, alkyl, alkoxy, thioalkoxy, halo, hydroxy or dihydrogenphosphate;
R₆ is H or alkyl;
R₇ is H or alkyl;
R₈ is hydrogen, alkoxy or dialkylaminoalkyl;
R₉ is optionally substituted alkyl;
R₁₀ is hydrogen, alkyl, alkoxy or dialkylaminoalkyl;
R₁₁ and R₁₂ are independently hydrogen, alkyl, alkoxy, thioalkoxy, amino, alkylamino, dialkylamino, morpholino, alkylmorpholino, piperidino, alkylpiperidino, piperazino, alkylpiperazino or 1-aziridinyl; and
A, together with the carbon atoms to which it is fused, is an optionally substituted aryl or heteroaryl ring.

2. A compound according to claim 1, wherein the alkyl groups in the R₁₋₇ and R₉₋₁₂ substituents are unsubstituted or substituted with 1, 2 or 3 unsubstituted substituents chosen from halogen, amino, mono(C₁-C₄ alkyl)amino, di(C₁-C₄ alkyl)amino, hydroxy, C₁-C₄ alkoxy and C₁-C₄ alkylthio substituents.

3. A compound according to claim 1, wherein the aryl and heteroaryl groups in the Ar, A and R₁ substituents are unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

4. A compound according to claim 1, wherein R₁ is hydrogen, unsubstituted C₁-C₆ alkyl, a phenyl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy or a heteroaryl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy substituents.

5. A compound according to claim 1, wherein R₁ is hydrogen or unsubstituted C₁-C₂ alkyl.

6. A compound according to claim 1, wherein R₂ is unsubstituted C₁-C₆ alkyl, unsubstituted C₁-C₄ alkoxy, unsubstituted thio(C₁-C₄ alkoxy) or a halo group.

7. A compound according to claim 1, wherein R₂ is an unsubstituted C₁-C₂ alkoxy group.

8. A compound according to claim 1, wherein R₃, R₄ and R₅ are the same or different and each represent unsubstituted C₁-C₆ alkyl, unsubstituted C₁-C₄ alkoxy, unsubstituted thio(C₁-C₄ alkoxy) or a halo group provided that at least two of R₃, R₄ and R₅ are alkoxy.

9. A compound according to claim 1, wherein R₃, R₄ and R₅ are the same or different and each represent unsubstituted C₁-C₂ alkoxy.

10. A compound according to claim 1, wherein X is O, S or NR₇, wherein R₇ is hydrogen or unsubstituted C₁-C₆ alkyl.

11. A compound according to claim 1, wherein X is O, S or NH.

12. A compound according to claim 1, wherein L is —OC(O)— or —OP(O)(OR₆)—, wherein R₆ is hydrogen or unsubstituted C₁₋₆ alkyl.

13. A compound according to claim 1, wherein L is —OC(O)—.

14. A compound according to claim 1, wherein n is 0.

15. A compound according to claim 1, wherein Y is selected from hydrogen, unsubstituted C₁-C₆ alkyl, unsubstituted C₁-C₄ alkoxy, unsubstituted thio(C₁-C₄ alkoxy), halo, hydroxy and dihydrogenphosphate substituents.

16. A compound according to claim 1, wherein Y is hydrogen.

17. A compound according to claim 1, wherein Ar is a substituted aryl or 5 to 10 membered heteroaryl group which carries one substituent selected from a nitro or azido group and 0, 1 or 2 further unsubstituted substituents chosen from halogen, C₁-C₆ alkyl, hydroxy, amino, C₁-C₄ haloalkyl, C₁-C₄ alkoxy and C₁-C₄ haloalkoxy substituents.

18. A compound according to claim 1, wherein Ar is an unsubstituted nitrophenyl, unsubstituted nitroimidazole, unsubstituted nitrothiophene or unsubstituted nitrofuranyl group.

19. A compound according to claim 1, wherein Ar is a group of formula (3), wherein R₉ is an unsubstituted C₁-C₆ alkyl group.

20. A compound according to claim 19, wherein R₉ is an unsubstituted C₁-C₂ alkyl group.

21. A compound according to claim 1, wherein R₁₀ is selected from hydrogen, unsubstituted C₁-C₆ alkyl, unsubstituted C₁-C₄ alkoxy and unsubstituted di(C₁-C₆ alkyl)amino(C₁-C₆ alkyl) substituents.

22. A compound according to claim 21, wherein R₁₀ is an unsubstituted C₁-C₂ alkyl group.

23. A compound according to claim 1, wherein R₁₁ and R₁₂ are the same or different and each represent an unsubstituted substituent selected from hydrogen, C₁-C₆ alkyl, C₁-C₄ alkoxy, thio(C₁-C₄ alkoxy), amino, (C₁-C₆ alkyl)amino, di(C₁-C₆ alkyl)amino, morpholino, (C₁-C₆ alkyl)morpholino, piperidino, (C₁-C₆ alkyl)piperidino, piperazino, (C₁-C₆ alkyl)piperazino and 1-aziridinyl substituents.

24. A compound according to claim 23, wherein R₁₁ and R₁₂ are the same or different and each represent a substituent selected from hydrogen, unsubstituted C₁-C₂ alkoxy and unsubstituted (C₁-C₂ alkyl)piperidino substituents.

25. A compound according to claim 1 which is 1-(4-methoxy-3-(5-nitrothien-2-yl)methoxy)phenyl-2-(3,4,5-trimethoxy) phenyl-Z-ethene, 1-(4-Methoxy-3-(1-(5-nitrothien-2-yl)ethoxy))phenyl-2-(3,4,5-trimethoxy)phenyl-Z-ethene, 1-(4-Methoxy-3-(5-nitrothien-2-yl) methoxycarbonyloxy)phenyl-2-(3,4,5-trimethoxy)phenyl-Z-ethene, 5-Methoxy-3-((3,4,4′,5-tetramethoxy-(Z)-stilbene-3′-yl)oxy)methyl-1,2-dimethylindole-4,7-dione or 3-((3,4,4′,5-Tetramethoxy-(Z)-stilbene-3′-yl)oxy)methyl-1,2-dimethyl-5-(4-methylpipe razin-1-yl)indole-4,7-dione, or a pharmaceutically acceptable salt thereof.

26. A pharmaceutical composition comprising a compound according to claim 1, or a pharmaceutically acceptable salt thereof, and a pharmaceutically acceptable carrier or diluent.

27. A method of ameliorating or reducing the incidence of a proliferative disorder in a patient, which method comprises administering to said patient an effective amount of a compound as defined in claim 1, or a pharmaceutically acceptable salt thereof.

28. (canceled)

29. A method according to claim 27, wherein the proliferative disorder is cancer, rheumatoid arthritis, psoriatic lesions, diabetic retinopathy or wet age-related macular degeneration.

30. A method according to claim 27, wherein the proliferative disorder is a hypoxic disorder.

31. A method according to claim 27, wherein the medicament is for use in the prevention or treatment of a solid tumour or leukaemia.

32. (canceled)

33. A method according to claim 30, which method comprises administering to said patient an effective amount of:
(a) a compound as defined in claim 1, or a pharmaceutically acceptable salt thereof; and
(b) a reductase, an anti-body reductase conjugate, a macromolecule-reductase conjugate or DNA encoding a reductase gene.

34. (canceled)
